(19) United States
(12) Patent Application Publication
(10) Pub. No.: US 2011/0053241 A1
(43) Pub. Date: Mar. 3, 2011

(54) DEVICE AND METHOD FOR MECHANICALLY DEFORMING CELLS

(75) Inventors: Jacob M.J. Den Tooner, Eindhoven (NL); Murray F. Gillies, Eindhoven (NL)

(73) Assignee: KONINKLIJKE PHILIPS ELECTRONICS N.V., EINDHOVEN (NL)

(21) Appl. No.: 12/935,028

(22) PCT Filed: Mar. 31, 2009
(86) PCT No.: PCT/IB2009/051355

§ 371 Date: Nov. 17, 2010

(30) Foreign Application Priority Data
Apr. 4, 2008 (EP) 08154094.0

(51) Int. Cl. C12N 13/00; C12M 1/00; C12M 1/34; C40B 60/00; C12N 5/00

(52) U.S. Cl. 435/173.1; 435/283.1; 435/287.1; 435/288.7; 506/33; 435/325

(57) ABSTRACT

The present invention relates to a device for mechanically deforming cells comprising: a cell holding element (33) for holding a cell (32) in a cell holding zone, a micro-actuator (31) for applying a force on the held cell (32), wherein said micro-actuator (31) can be electrically, thermally, photonically or magnetically actuated and wherein the micro-actuator (31) applies said force on the cell (32) in a non-actuated or an actuated state, and a stimulation unit (35) for electrically, thermally, photonically or magnetically actuating said micro-actuator (31).

### FIELD OF THE INVENTION

The present invention relates to a device and a corresponding method for mechanically deforming cells.

### BACKGROUND OF THE INVENTION

The past decade has seen substantial growth in research into how changes in the biochemical and biophysical properties of cells and subcellular structures influence and are influenced by the onset and progression of human diseases. It has been found in particular that the mechanical properties of cells are relevant for many diseases including cancer and coronary artery disease: diseases which cause large numbers of fatalities in the western world. According to the World Health Organization 7.6 million people worldwide died of cancer in 2005, while 8 million people in the US had a myocardial infarction. Other diseases that have been shown to influence the mechanical properties of the cell are malaria, cardiac myopathy, and muscular dystrophies.

The change in mechanical properties due to a disease can be significant. The elastic modulus of a cancerous cell can be an order of magnitude smaller than that of a healthy cell.

This indicates that the mechanical properties of cells in particular their stiffness is indeed a sensitive marker for the progression and/or presence even in the initial stages of a range of diseases. By measuring the cell stiffness it is in principle possible to detect single malignant cells and precancerous cells—malignantly transformed cells are easier to deform—and this opens for example the possibility to monitor the progress of cancer from pre-invasive to invasive. Similar arguments hold for the other diseases mentioned earlier.

Several approaches are being used to measure the mechanical properties of cells. Some of these techniques probe local deformation properties of the cell for example partial micropipette aspiration, cell indentation, and atomic force microscopy, while others probe the cell as a whole e.g. full micropipette aspiration, magnetic bead twisting, and cell compression testing. These measurement methods have various disadvantages.

First, for the local methods the response may depend significantly on the precise probing location and thus show a large cell-to-cell spread. Second, most methods are tedious and very time-consuming and are therefore not suitable for use in rapid clinical diagnosis. Third, other methods have the inherent risk of imposing damage to the cells (e.g. optical stretchers), leading to a faulty measurement. Finally, in many methods it is impossible to create a suitable environment around the cells to be tested leading to rather artificial and irrelevant results.

### SUMMARY OF THE INVENTION

It is an object of the present invention to provide a device and a corresponding method for mechanically deforming cells which avoid the above described disadvantages, and which preferably allow to monitor the mechanical properties of single cells but also of many cells simultaneously.

In a first aspect of the present invention a device for mechanically deforming cells is presented comprising
a cell holding element for holding a cell in a cell holding zone,
a micro-actuator for applying a force on the held cell, wherein said micro-actuator can be electrically, thermally, photonically or magnetically actuated and wherein the micro-actuator applies said force on the cell in a non-actuated or an actuated state, and
a stimulation unit for electrically, thermally, photonically or magnetically actuating said micro-actuator.

In a further aspect of the present invention a corresponding method is presented comprising the steps of
holding a cell in a cell holding zone, and
electrically, thermally, photonically or magnetically actuating a micro-actuator for applying a force on the held cell, wherein said micro-actuator can be electrically, thermally, photonically or magnetically actuated and wherein the micro-actuator applies said force on the cell in a non-actuated or an actuated state.

Preferred embodiments of the invention are defined in the dependent claims. It shall be understood that the claimed device and the claimed method have similar and/or identical preferred embodiments as defined in the dependent claims.

The present invention is based on the idea to hold one (or more) cell(s) in one (or more) cell holding position(s) and to deform said one (or more) cells by applying a mechanical force by use of one (or more) micro-actuator(s), for instance to probe the mechanical properties of said cell(s). For this purpose different types of micro-actuators can be used, for instance polymer actuators, which have been described in WO 2006/087655 A1 and WO 2008/020374 A2 for manipulation (transportation, mixing, routing) of fluids in micro-fluidic devices.

According to a preferred embodiment, the micro-actuator has the form of a stripe which is curled in one of non-actuated or actuated state and non-curled in the other state as for instance disclosed in the above mentioned prior art documents. In one embodiment the force is applied to the cell when the micro-actuator is actuated from the curled state into the rolled out state whereas in another embodiment the force is applied to the cell when the micro-actuator is released from the actuated (rolled out) state into the curled state. In further embodiments the micro-actuator can also be adapted such that the curled state is the actuated state and that the rolled out state is the non-actuated state.

According to a further embodiment the micro-actuator comprises a double-layer including a polymer film layer in particular an acrylate film and an electrically conductive film layer and the stimulation unit comprises a stimulation electrode and a voltage source for applying a voltage between said stimulation electrode and said conductive film layer. In particular a polymer MEMS (micro-electro-mechanical system) actuator (PMA) as described in WO 2008/020374 A2 is employed according to this embodiment which is easily to control by application of a voltage, e.g. a high AC voltage.

According to an alternative embodiment the micro-actuator comprises a magnetic material and the stimulation unit comprises a magnetic field unit for generating a magnetic field through said cell holding zone. In this embodiment the micro-actuator preferably comprises a composite structure, in particular a polymer film with dispersed magnetic particles or a stack of non-magnetic and a magnetic films. The advantage of magnetic stimulation and detection over electrical stimulation and detection is that the magnetic field exhibits less interaction with biological materials present in the device. Therefore, electrochemical effects such as electrolysis are avoided easily and the detection is less influenced by background noise.

To enable a monitoring of the mechanical properties of cells a sensing element is provided adjacent to said cell holding zone for sensing the deformation of the cell when a force is applied to the cell by said micro-actuator. Thus the deformation (in particular the amount, location, duration, etc. thereof) of the cell induced by the force applied by the micro-actuator and hindered by the stiffness of the cell is detected allowing to get significant information about the cell.

According to a preferred embodiment the sensing element is an optical magnetic or electric sensing element in particular a camera, a GMR sensor or a sensing electrode. Thus different options exist for detection of the deformation. Optical sensing has the advantage of being a straight-forward commonly used approach for cell imaging. Electrical sensing has the advantage of enabling integration in the device.

Still further sensing element preferably comprises a sensing electrode and a capacitance measuring element for measuring the capacity between said sensing electrode and said conductive film layer of said micro-actuator. In particular in embodiments using electrostatic actuation the capacitance measurement is preferred the capacitance being a measure for the distance between the sensing electrode and the actuator electrode (conductive film layer of the actuator).

A further application of the device and method of the present invention is the lysing of cells. For this purpose according to an embodiment the stimulation unit is adapted for applying a stimulation signal, which is so large that the micro-actuator applies a force on the cell causing the cell to lyse. This provides a simple and effective possibility of lysing cells.

To avoid that the cell is pushed away from the cell holding position by the application of force from a single actuator, it is proposed according to a further embodiment that two or more micro-actuators are arranged on different sides of the cell holding element for applying a force on the same cell from different directions.

In a further embodiment an array of micro-actuators and associated cell holding elements are provided for simultaneously deforming a number of cells. In this way statistics of the mechanical properties of a large number of cells can be obtained quickly. For this purpose, an LTPS (low temperature polycrystalline Si) platform, as for instance described in WO 2008/020374 A2, can be used, according to which a number of micro-actuators is arranged in a two-dimensional matrix array.

Preferably the invention is used a micro-fluidic system and comprises a micro-fluidic chamber including said micro-actuator, said cell holding element, said stimulation unit and a buffer solution, in particular a sugar solution, containing the cells. Such micro-fluidic systems are in general also described in WO 2006/087655 A1 and WO 2008/020374 A2.

### BRIEF DESCRIPTION OF THE DRAWINGS

These and other aspects of the invention will be apparent from and elucidated with reference to the embodiment(s) described hereinafter. In the following drawings:

FIG. 1a-1e shows various embodiments of a micro-actuator used according to the present invention,

FIG. 2 shows the general layout of an embodiment of the device according to the present invention,

FIG. 3a-3b shows a first embodiment of a device according to the present invention,

FIG. 4a-4b shows a second embodiment of a device according to the present invention,

FIG. 5 shows a third embodiment of a device according to the present invention, and

FIG. 6 shows the general layout of an array of micro-actuators.

### DETAILED DESCRIPTION OF THE INVENTION

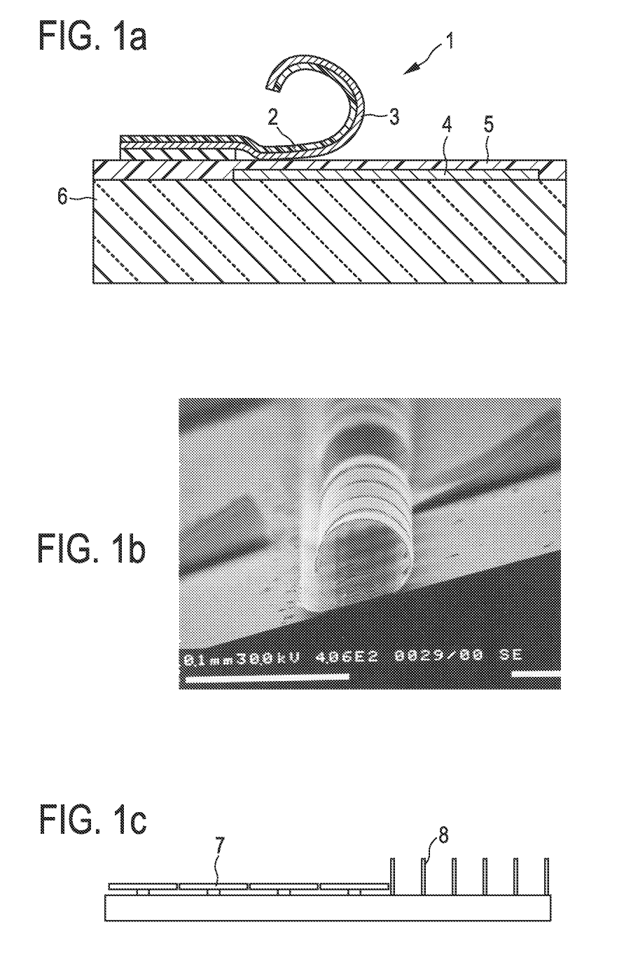

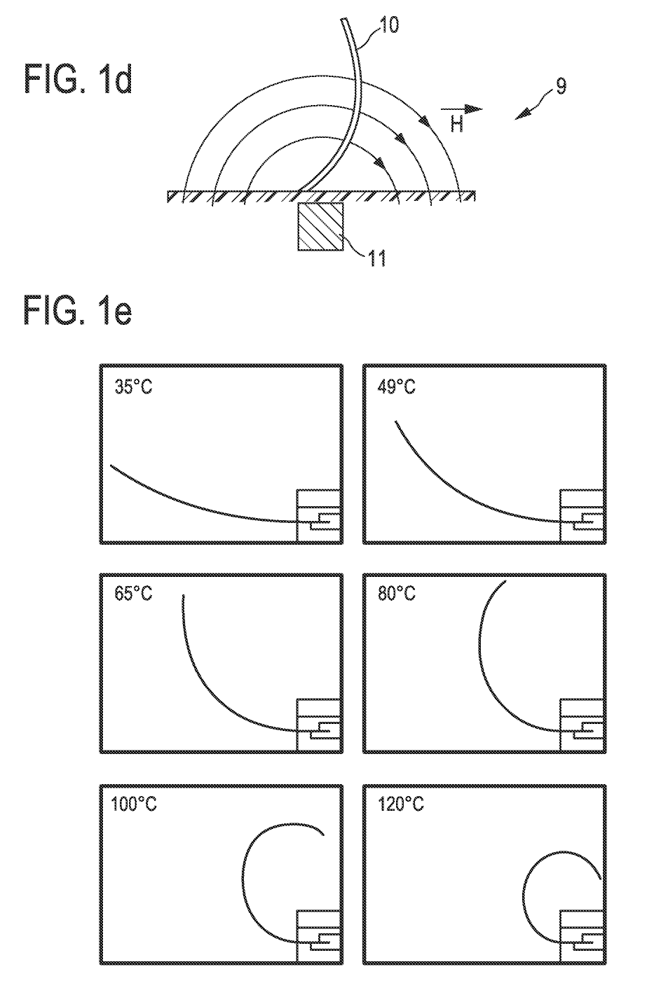

FIG. 1 shows a number of embodiments of a micro-actuator. FIG. 1a shows a double layer composite structure of a micro-actuator 1 comprising a polymer film 2 (e.g. an acrylate) and an electrically conductive film 3 (e.g. chromium). The processing is tuned such that the structure curls upward being attached at one end. When a voltage difference is applied between an electrode 4, placed underneath the actuator 1 and insulated from the conductive film 3 by another insulating layer 5 (e.g. an acrylate layer) and the conductive film 3, an electrostatic force will pull the actuator 1 towards the substrate 6. Consequently, it will roll out and flatten out on the substrate 6. When the voltage is removed the slab will return to its original curled shape by elastic recovery. The actuation effect is bi-stable and the position of the actuator tip is a function of the applied voltage. For a particular PMA design the “unroll” voltage Vun is 11 V, and the “elastic recovery” voltage Ver is 5V. These values can be tuned typically between 1V and 100V depending on the dimensions and mechanical properties of the actuator 1. FIG. 1b shows a SEM picture of actual structures made in this case with a length of 100 μm, a width of 20 μm, and a thickness of 1 μm. Such an embodiment of an actuator is described in more detail in WO 2008/020374 A2 (cf. FIG. 1).

Many alternatives to the geometry shown in FIGS. 1a and 1b are conceivable. Instead of curled strips they may be straight beams, cylindrical rods, and so forth. The initial orientation of the strips 7, 8 may be parallel (strips 7) or perpendicular (strips 8) to the surface, as illustrated in FIG. 1c.

Further “stimuli” other than electrical field may be used to actuate the structures. A magnetically stimulated actuator 9 having an actuator strip 10 is depicted in FIG. 1d. The actuator strip 10 consists of a composite material, of which one component is magnetic. One example is a polymer film with dispersed magnetic particles. The latter may be paramagnetic or ferromagnetic. Another example is a structure consisting of a stack of non-magnetic (e.g. polymer) and a magnetic (e.g. nickel) films. Such a magnetic actuator can be set into motion by magnetic field that is generated by external means, such as (a combination of) coils, or by integrated current wires or coils, as is illustrated in FIG. 1d where a current wire 11 indices a magnetic field near the strip 10, which then moves due to the magnetic force acting upon it. Such an embodiment of an actuator is described in more detail in WO 2006/087655 A1 (cf. FIG. 13).

Even other possibilities are actuators responding to light or temperature. Several polymeric materials that respond to a change in temperature by deforming are known. An overview and some background can be found in Broer et al. [Dirk J. Broer, Henk van Houten, Martin Ouwerkerk, Jaap M. J. den Toonder, Paul van der Sluis, Stephen I. Klink, Rifat A. M. Hikmet, Ruud Balkenende. Smart Materials. Chapter 4 in True Visions: Tales on the Realization of Ambient Intelligence, ed. by Emile Aarts and José Encarnaçao, Springer Verlag, 2005.] For example by incorporating LC (liquid crystalline) material into an elastomeric network a material can be made which upon heating through a specific temperature (the nematic isotropic temperature) undergoes a transition in the backbone of the elastomer molecules and changes length. By careful control of processing conditions [D. J. Broer et al, accepted for pub. in Adv. Funct. Mater., (2005)] it is possible to obtain a gradient in orientation of LC molecules over the thickness of the film so that one side of the film contracts while the other expands. This creates a reversible rolling up of the film at a specific temperature. FIG. 1e shows cross-sectional photos of such a film at various temperatures.

Light-actuation or photonic addressing can be achieved using photoresponsive materials, containing chromophores, leading to photochromism. Photochromism is defined as a reversible phototransformation of a chemical species between two forms with different absorption spectra. During the photoisomerisation, also other properties may change, such as the refractive index, dielectric constant and geometrical structure. Particular non-limitative examples of these materials include azobenzenes, spirobenzopyranes, stilbenes, α-hydrazono-β-ketoesters, and cinnamates.

The polymer-based actuators can be integrated in a micro-fluidic system, for example covering the floor of a micro-fluidic chamber or channel in an arrayed arrangement. In the case of electrostatic, magnetic, or temperature-actuation, the electrode pattern can be designed and manufactured such that the micro-actuators or groups of them can be addressed individually.

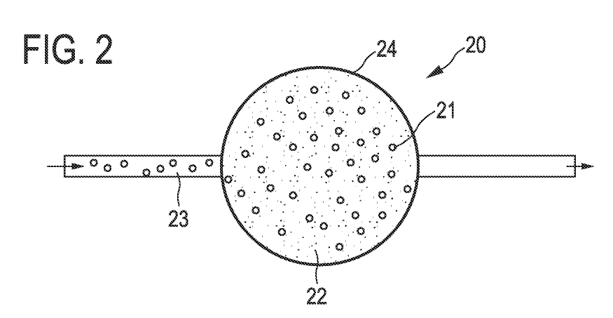

According to an application of the present invention a single micro-actuator or an array of micro-actuators is integrated in micro-fluidic systems to measure the mechanical properties in particular the stiffness of biological cells (e.g. for diagnostic analysis). The key is to trap/tether the cells on top of the micro-actuator(s) or between them apply a force on the cells through actuating the micro-actuator(s), and detecting the deformation of the micro-actuator(s), which will be induced by the stimulus such as electrical field or magnetic field but hindered by the stiffness of the attached/contacting cell. The general layout of an embodiment 20 of the device according to the present invention is as shown in FIG. 2. The cells 21, suspended in a buffer liquid 22, are supplied through a supply channel 23 into a diagnostic chamber 24. The chamber 24 contains cell trapping sites and corresponding polymer actuators (both not shown in FIG. 2). The cells 21 are deformed using the actuators, while the level of deformation is sensed. In such a diagnostic chamber 24 using an array of micro-actuators many cells 21 can be tested simultaneously.

In the following a number of particular embodiments will be shown and explained to illustrate the present invention in more detail.

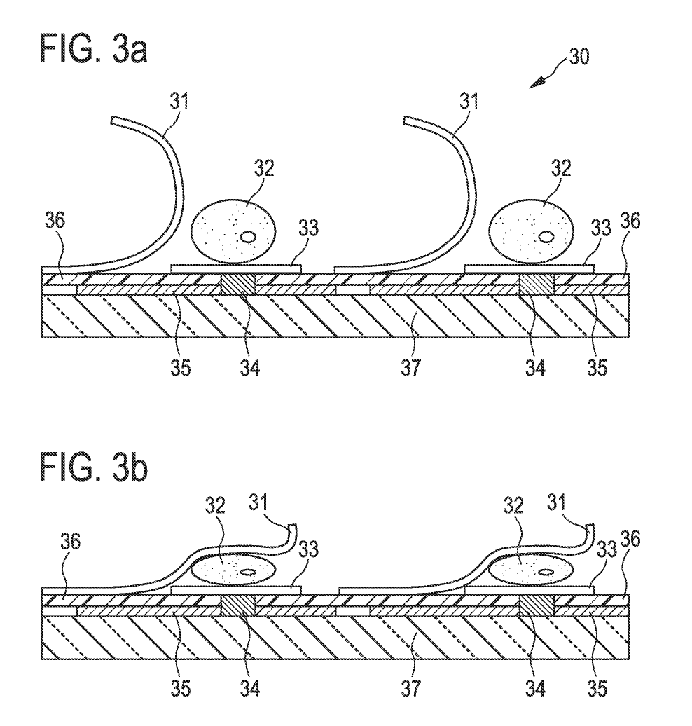

One main application of the present invention is cell stiffness measurement using cell squeezing by actuated micro-actuators. A first embodiment of a device 30 for such an application is shown in FIG. 3. This embodiment comprises two micro-actuators 31 for separately deforming a single cell 32, which is held in a cell holding position by a cell holding element 33. Below the cell holding elements 33 sensing units 34, e.g. sensing electrodes 34, are provided for measuring the deformation of the cell 32 above it when a force is applied to the cell 32 by the respective micro-actuator 31. Further, an actuating electrode 35 is provided below each of the micro-actuators 31, which is insulated from the (conductive) micro-actuator 31 by an insulating layer 36. All elements are provided on a substrate 37.

The micro-actuators 31 are similar to those shown in FIGS. 1a, 1b. They could be electrostatically actuated or magnetically actuated structures. In the non-actuated state shown in FIG. 3a, they are curled away (upwards) from the substrate 37. The cells 32 are trapped between the actuators 31, on the cell adhesion spots by the cell holding elements, which are, for example, formed by cell adhesion proteins (integrins). Alternatively, tissue adhesives such as BD Cell-Tak™ can be placed at the cell holding positions as cell holding elements 33.

The size and spacing of the actuators 31 should be tuned to the cell size. Since a typical biological cell size is 10 to 20 μm, the size and spacing of the actuators 31 should be several tens of μm, which is easily achievable with the current technology.

Preferably a high frequency AC voltage is applied to the actuating electrodes 35 to roll-out the flap of the micro-actuators 31, as shown in FIG. 3b. The same signal can also be used for probing the impedance of the overlying flap and therefore used to sense the position of the flap and deduce the presence, and eventual size, of a trapped cell 32.

The cell 34 should be situated directly on top of the sense electrode 34, and there should preferably be a gap in the insulator 36 so that the actuating electrodes 35, makes direct contact with the medium in which the cells 32 are situated. This concentrates the field lines through the cells 32 and increases the sensitivity of the electrical measurement.

In a slightly modified embodiment, the size of the micro-actuators is such that it is possible to have multiple sense electrodes 34 under each flap.

When actuated, the micro-actuators 31 are attracted towards the substrate 37 and the cells 32 are “squeezed”. The resulting deformation of the cell 32 and the corresponding shape change of the actuators 31, is determined by the cell stiffness. The deformation may be observed in various ways:

i) optically, e.g. by direct imaging with a CCD

ii) magnetically: if the actuators 31 are magnetic, a magnetic detector (as sensing element 34) integrated in the substrate 37, e.g. a GMR sensor, can detect the movement and global shape of the actuator 31;

iii) from capacitance measurements (in particular for electrostatic actuation): the capacitance between the electrode integrated in the actuator 31 and actuating electrode 35 integrated in the substrate 37 depends on the distance between them; measurement of this capacitance, hence, gives information about the extent of squeezing of the cell 31. In practice it will probably be most interesting to first apply a voltage to induce actuator roll-out. The capacitance immediately after roll-out is a measure of the volume of the cell 31 trapped under the flap. The voltage and therefore the force applied can then be ramped and the capacitance measured. This gives a deformation as a function of force curve.

The forces that would be necessary to deform the cell significantly are in the order of 1 nN, and these values can be easily reached with the proposed electrostatic or magnetic actuators.

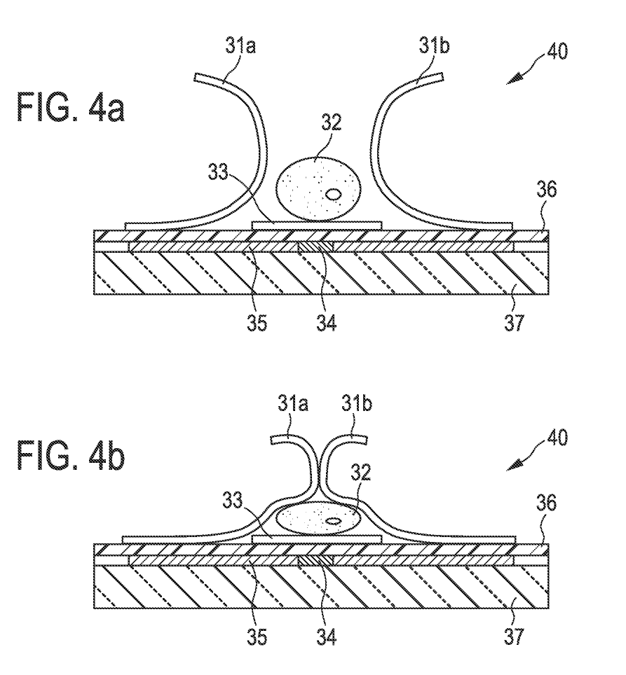

An alternative embodiment of a device 40 according to the present invention is shown in FIG. 4. In this embodiment two micro-actuators 31a, 31b are provided per cell holding position located on opposite sides of the cell holding element 33. FIG. 4a again shows the non-actuated state, FIG. 4b shows the actuated state. As can be seen from FIG. 4b the cell 32 is squeezed from two sides, decreasing the possibility that it is pushed away from the cell adhesion spot instead of being deformed. It shall be noted that, of course, also more than two micro-actuators 31a, 31b can be positioned around single cell adhesion spot to further increase this advantage.

In a further embodiment an electrically active substrate is used. Then it is also possible to design electrode geometries on the substrate which locate the cell at the required location. This can be in the form of a hole in the actuating electrode or any low E-field trap and can be used for either holding the cell or for manipulating it into the correct location for binding with the integrins.

The holding mechanism for the cell can also be of a microfluidic origin where a small hole is created between two volumes. A pressure difference between the volumes will suck the cells into the hole and hold the cell for probing.

For actuating it is proposed in an embodiment to place the cells in a sugar (sucrose or mannitol) water buffer solution. This medium has a low electrical conductivity and therefore prevents any ionic shielding of the electrical fields.

Another main application of the present invention is clean mechanical lysing. If the cell is firmly held on the adhesion spot, then the actuating voltage can be intentionally set very high. This results in the flap being actuated with an enormous force and can result in the lysing of the trapped cell. This is interesting as the cell membrane is thereafter bound to the substrate while the contents of the cell are free to diffuse into the solution. This is desirable for single cell PCR (Polymerase Chain Reaction) or for any integrated bio device where downstream DNA extraction has to be performed.

The invention can also be used with magnetic actuation and detection. As shown in FIG. 1d current wires are integrated in the substrate. Running a current through them generates a concentric magnetic field that attracts the actuators toward the surface.

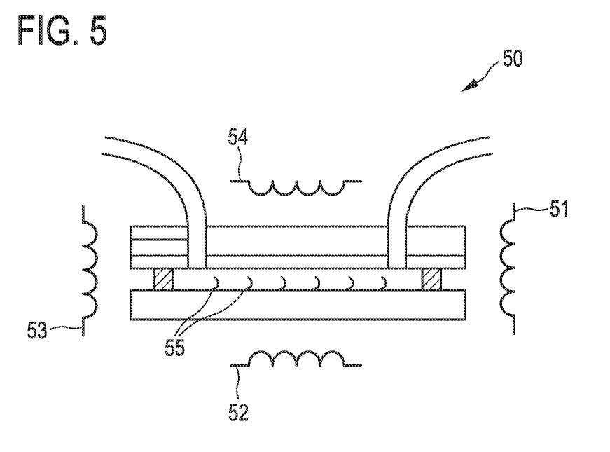

Another possibility is to place electromagnets or magnetic coils around the device, for example four magnetic coils 51-54 in a symmetric layout of a microfluidic device 50 illustrated in FIG. 5. The magnetic coils 51-54 can be individually addressed. It will be possible to generate a magnetic field that changes in time and in magnitude, by which the actuators 55 (polymer micro actuators) are stimulated.

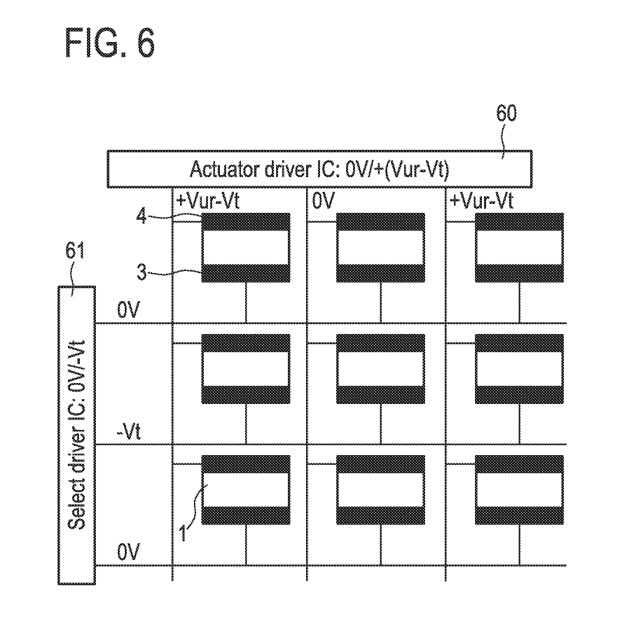

The general layout of an array of micro-actuators is shown in FIG. 6. The array of electrodes 3, 4 of the micro-actuators 1 can be connected to external voltage drivers 60, 61. In order to realize this passive matrix layout, it is necessary that both the actuation and foil electrodes are structured in the form of lines orientated at an angle to each other. In the example of FIG. 6, the actuation electrodes have been structured in the form of columns, whilst the foil electrodes 3 have been structured in the form of rows. In order for a passive matrix system to operate successfully, it is required that the micro-actuator 1 exhibits a voltage threshold. A voltage of around Vur is required to unroll the foil 3, whereby a voltage of around Vt will be insufficient to initiate the unrolling.

Each row and each column can be individually attached to a voltage source. For example, the row electrodes (foil electrodes 3) may be connected to a select driver 61, e.g. a standard-shift register similar to a gate driver for an AMLCD, which can switch between 0V and Vt. The column electrodes (actuator electrodes 4) are then connected to the actuation driver 60. The actuation driver 60 could be just a standard voltage data driver as used for e.g. passive or active matrix liquid crystal displays (LCD), with outputs which may have either 0V or (Vur−Vt) levels.

The operation of this array and further embodiments of arrays of micro-actuators which can generally be employed according to the present invention are shown in FIGS. 2-6 of WO 2008/020374 A2, the description of which being incorporated herein by reference.

Thus, according to the present invention it is possible to obtain statistics of the cell property measured since the signal can be read out per individual actuator. The use of an LTPS platform as described for instance in WO 2008/020374 A2 enables this. Alternatively the actuators could also be grouped together to give one average figure for the population.

Further, the actuation can be done in a dynamic time-varying way to probe time-dependent mechanical properties of cells. The method can also be combined with a cell sorting method. Still further, the “environment” (chemical, temperature) of the cell can be controlled to create either special or optimal conditions.

There are different fields of application of the invention:
Mechanical characterization of cells in general;
Mechanical lysis of cells;
Diagnostic micro-fluidic device for cancer, malaria, cardiac myopathy, muscular dystrophies or other diseases that affect the cell's mechanical properties: detection of presence or progression of these diseases;
Screening large amounts of cells for affected cells e.g. when trying to find a few cancer cells among many normal cells;
Screening for the effect of pharmaceuticals.
(Simultaneous) Measurement of mechanical properties of (many) cells using micro-actuators integrated in a micro-fluidic system; the method enables to obtain statistics of the property of interest since the read-out can be done in principle per actuator, e.g. using an LTPS platform;
A medical diagnostic device on the basis of this principle;
Electrostatic/magnetic/optical/thermal actuation in combination with electrostatic/magnetic/optical detection.

In conclusion an aim of the present invention is to provide a device and a method to determine the mechanical properties of biological cells by deforming them using micro-actuators integrated in a micro-fluidic device. The method is such that many cells may be analyzed simultaneously. Since the mechanical properties of cells are relevant for many diseases including cancer and coronary artery disease the proposed micro-fluidic device may be used as a fast and sensitive diagnostic tool for detecting the presence or progression of these diseases. Further, lysing of cells is possible.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measured cannot be used to advantage.

Any reference signs in the claims should not be construed as limiting the scope.

1. Device for mechanically deforming cells comprising:
a cell holding element (33) for holding a cell (32) in a cell holding zone,
a micro-actuator (31) for applying a force on the held cell (32), wherein said micro-actuator (31) can be electrically, thermally, photonically or magnetically actuated and wherein the micro-actuator (31) applies said force on the cell (32) in a non-actuated or an actuated state, and
a stimulation unit (35) for electrically, thermally, photonically or magnetically actuating said micro-actuator (31).

2. Device as claimed in claim 1,
wherein said micro-actuator (31) has the form of a stripe, which is curled in one of non-actuated or actuated state and non-curled in the other state.

3. Device as claimed in claim 1,
wherein said micro-actuator (31) comprises a double-layer including a polymer film layer (2), in particular an acrylate film, and an electrically conductive film layer (3), and
wherein said stimulation unit (35) comprises a stimulation electrode (4) and a voltage source (60) for applying a voltage between said stimulation electrode (4) and said conductive film layer (3).

4. Device as claimed in claim 1,
wherein said micro-actuator (31) comprises a magnetic material, and
wherein said stimulation unit (35) comprises a magnetic field unit for generating a magnetic field through said cell holding zone.

5. Device as claimed in claim 4,
wherein said micro-actuator (31) comprises a composite structure, in particular a polymer film with dispersed magnetic particles or a stack of non-magnetic and a magnetic films.

6. Device as claimed in claim 1,
further comprising a sensing element (34) adjacent to said cell holding zone for sensing the deformation of the cell (32), when a force is applied to the cell (31) by said micro-actuator (31).

7. Device as claimed in claim 6,
wherein said sensing element (34) is an optical, magnetic or electric sensing element, in particular a camera, a GMR sensor or a sensing electrode.

8. Device as claimed in claim 3,
wherein said sensing element (34) comprises a sensing electrode and a capacitance measuring element for measuring the capacity between said sensing electrode and said conductive film layer of said micro-actuator.

9. Device as claimed in claim 1,
wherein said stimulation unit (35) is adapted for applying a stimulation signal, which is so large that the micro-actuator applies a force on the cell causing the cell to lyse.

10. Device as claimed in claim 1,
comprising two or more micro-actuators (31a, 31b) arranged on different sides of the cell holding element (33) and adapted for applying a force on the same cell (32) from different directions.

11. Device as claimed in claim 1,
comprising an array of micro-actuators (31) and associated cell holding elements (33) for simultaneously deforming a number of cells (32).

12. Device as claimed in claim 1,
comprising a micro-fluidic chamber (24) including said micro-actuator (31), said cell holding element (33), said stimulation unit (35) and a buffer solution (22), in particular a sugar solution, containing the cells (21).

13. Method for mechanically deforming cells comprising the steps of:
holding a cell (32) in a cell holding zone, and
electrically, thermally, photonically or magnetically actuating a micro-actuator (31) for applying a force on the held cell (32), wherein said micro-actuator (31) can be electrically, thermally, photonically or magnetically actuated and wherein the micro-actuator (31) applies said force on the cell (32) in a non-actuated or an actuated state.
